(19) United States
(12) Patent Application Publication
(10) Pub. No.: US 2003/0013138 A1
(43) Pub. Date: Jan. 16, 2003

(54) Systems and methods for the analysis of proteins

(75) Inventors: Samir Hanash, Ann Arbor, MI (US); Francois LeNaour, Villejuif (FR); Anne Marie Yim, Ann Arbor, MI (US); Ashkan Haghighat, Montreal (CA); Franck Brichory, Cruseilles (FR)

Correspondence Address: MEDLEN & CARROLL, LLP, Suite 350, 101 Howard Street, San Francisco, CA, 94105, US

(73) Assignee: The Regents of the University of Michigan, Ann Arbor, MI

(21) Appl. No.: 10/157,053

(22) Filed: May 29, 2002

Related U.S. Application Data
(60) Provisional application No. 60/294,120, filed on May 29, 2001.

(51) Int. Cl. G01N 33/567; G01N 33/53

(52) U.S. Cl. 435/7.21; 435/7.5

(57) ABSTRACT

The present invention relates to systems and methods for the analysis of proteins. For example, the present invention provides methods for identifying and characterizing surface membrane proteins. The present invention also provides methods and systems for arraying and analyzing proteins.

The present application claims priority to U.S. Provisional Appln. No. 60/294,120, filed May29, 2001. This invention was made in part during work partially supported by PHS grant CA 84982. The U.S. government has certain rights in this invention.

### FIELD OF THE INVENTION

The present invention relates to systems and methods for the analysis of proteins. For example, the present invention provides methods for identifying and characterizing surface membrane proteins. The present invention also provides methods and systems for arraying and analyzing proteins.

### BACKGROUND OF THE INVENTION

Proteomics is an emerging field aimed at combining several technologies for the purpose of identifying the protein constituents of living organisms and the way they interact, and for determining their patterns of expression and post-translational modification in health and in disease and in response to exogenous factors. The justification of this effort is that proteins represent the most functional compartment of a cell and the information obtained at the protein level cannot simply be predicted from deciphering an organism's genome or by examining expression at the RNA level. The proteomic approach uniquely captures the contribution of post-translational protein modifications to cell function. The current interest in proteomics stems from the availability of methods to quantitatively analyze complex proteins mixtures, the availability of methods to identify proteins and their post-translational modifications, the development of bioinformatics tools to link protein and DNA sequences, and the capability to develop databases for storage and querying of protein information.

Proteome expression analysis typically involves a sequence of technologies that separate, map and then characterize proteins. The two most widely used technologies in contemporary proteomics are two-dimensional (2-D) electrophoresis for protein separation and mapping, and mass spectrometry (MS) for protein characterization. One of the problems with the use of 2-D gels to quantitatively analyze proteins in cell or tissue lysates is that 2-D gels can routinely resolve no more than 1,000-2,000 proteins because of limited resolution and sensitivity. A given cell type may express proteins derived from some 10,000 genes with a quite dynamic range of protein levels, which makes it difficult to visualize all but the most abundant proteins. Thus, there is currently a need to develop novel strategies for proteomics that provide substantially increased sensitivity for the quantitative analysis of low abundance proteins, without sacrificing the ability to undertake quantitative analysis of the remainder of proteins in the same cell or tissue sample.

### SUMMARY OF THE INVENTION

The present invention relates to systems and methods for the analysis of proteins. For example, the present invention provides methods for identifying and characterizing surface membrane proteins. The present invention also provides methods and systems for arraying and analyzing proteins.

For example, the present invention provides a method for identifying cell surface proteins comprising providing: a sample comprising one or more cells, said cells comprising surface proteins and intracellular proteins, and a non-membrane-permeable label (i.e., a label that membrane-impermeant, and when exposed to a cell surface, remains substantially only on the outside of the membrane and labels substantially only proteins on the outside of the membrane); exposing the sample to the label under condition such that the label binds to the surface proteins to generate labeled surface proteins; and identifying two or more of the labeled proteins (e.g., three or more, . . . ten or more, . . . 100 or more, . . . ). Thus, the present invention provides methods for simultaneously analyzing multiple membrane proteins or any type or class. In some embodiments, the analyzed proteins comprise different classes of proteins (e.g., proteins with different enzymatic activities than one another). For example, in some embodiments, the two or more identified labeled proteins comprise a first protein from a first class of proteins and a second protein from a second class of proteins, wherein the first class and the second class of proteins are different than one another and are from protein classes including, but not limited to, proteins kinases, growth factors, protein phosphatases, ion channels, and receptors.

In some embodiments, prior to the identifying step, the labeled surface proteins are separated from the intracellular proteins. In some such embodiments, the label comprises biotin (e.g., membrane-impermeant NHS-biotin). In some embodiments, biotin-labeled proteins are separated by binding the labeled surface proteins to a solid support comprising avidin.

The present invention is not limited by the nature of the protein identification or analysis. In some embodiments, the identifying step comprises mass spectrally analyzing the labeled proteins. In some embodiments, the method further comprises the step of quantitating an amount of at least one of the two or more identified labeled proteins. In some preferred embodiments, the method further comprises the step of comparing the amount of the identified labeled proteins to an amount of the same protein(s) or similar protein(s) from a different cell sample.

In some embodiments, prior to the identifying step, the labeled surface proteins are solubilized in a buffer. In some embodiments (e.g., where one or more of the labeled proteins is not solubilized in the buffer), prior to the identifying step and following the solubilizing step, the unsolubilized labeled proteins are digested.

Proteins may be derived from any desired cell samples, including but not limited to, cancer cells, undifferentiated cells (e.g., stem cells), differentiated cells, drug-treated cells, cell culture cells, tissue (e.g., animal or plant tissue), and the like.

The present invention also provides methods for arraying proteins, comprising, providing: a solid support, a sample comprising cellular proteins, and a separation apparatus that separates proteins based on a first physical property; treating the sample with the separation apparatus to produce a plurality of protein fractions; and attaching proteins from one (or more) of the protein fractions to a pre-selected location on the solid support. In some such embodiments, the proteins having at least one property in common are arrayed together. In some embodiments, a plurality of separation steps are carried out, each of which separates proteins based on one or more different physical properties. In such embodiments, proteins (e.g., cell surface proteins isolated by the methods described above) are grouped into a plurality of sub-groups defined by two or more criteria. The sub-groups of proteins may then be arrayed together in predetermined locations on a solid support.

Proteins arrayed by the methods of the present invention allow investigation and analysis or proteins with similar properties, apart from proteins that do not share the properties. For example, the methods of the present invention may be used to isolate and array proteins that are candidate targets for drug development. The protein array may then be used in drug screening, wherein all or many of the arrayed proteins (as opposed to few or none) are of the type of protein suitable for the drug assay. The methods of the present invention find particular use in the arraying and characterization of rare proteins, where, if they are arrayed among total cell protein, may not be present in sufficient quantity to distinguish their presence or behavior.

### DESCRIPTION OF THE FIGURES

FIGS. 1A, B, and C show separated proteins in some embodiments of the present invention. FIG. 1A shows a 2-D gel separation of whole cell proteins from A549 adenocarcinoma cell line. The gel is silver stained. First dimension separation using carrier ampholytes was carried out. Second dimension separation was conducting using 7-14% acrylamide gradient in SDS.

FIG. 1B shows a 2-D gel separation and blotting of whole cell proteins from A549 using the same conditions as FIG. 1A after biotinylation of surface membranes. Biotinylated proteins are visualized with streptavidin conjugates. FIG. 1C shows data similar to 1B, but of an independent experiment, showing the high degree of reproducibility of biotinylated protein patterns.

FIGS. 2A and B show separated proteins in some embodiments of the present invention. FIG. 2A shows a 2-D gel separation of A549 whole cell lysates, after biotinylation of surface membranes. The conditions were the same as in FIG. 1B except that an immobilized pH gradient (pH 3-10) was used in the first dimension of 2-D PAGE. Proteins were transferred onto a PVDF membrane and visualized by hybridization with a streptavidin conjugate. FIG. 2B shows immobilized pH gradient-based 2-D separation of biotinylated surface membrane proteins captured on avidin column and subsequently eluted. The proteins are visualized by silver staining, showing similarity to the pattern revealed in FIG. 2A.

FIG. 3 shows an analysis of modified Ag of cell surface proteins of A549 after immunoaffinity purification (7-14%) IPG-ASB 14 lysis buffer.

FIG. 4 shows a Western blot of biotinylated wce SYSY (7-14%)-IPG.

FIG. 5 shows a comparison of annexins I and II in the biotinylated surface membrane protein fraction (top panel) and in the whole cell lysate (bottom panel) of A549 cells. Purified surface membrane proteins (top panel) and whole cell lysates (bottom panel) were separated by IPG 2-D PAGE and annexins visualized with anti-annexin antibodies.

FIG. 6 shows a protein separation system in one embodiments of the present invention.

FIG. 7 shows a protein separation system in one embodiments of the present invention.

FIG. 8 shows a protein detection system in one embodiments of the present invention.

FIG. 9 shows a protein detection system in one embodiments of the present invention.

FIG. 10 shows an analysis of biotinylated surface membrane protein fractions following an ion exchange chromatography and SDS gel electrophoresis of individual fractions.

FIG. 11 shows a sample fractionation of A549 lung adenocarcinoma cells using a two-step separation method. The first separation is anion exchange, in which thirty fractions were collected. The second separation is reverse phase chromatography of an individual fraction from the first separation.

FIG. 12 shows a protein microarray experiment result in which 20 fraction of A549 lung adenocarcinoma cells were microarrayed in multiple patches per slide. Each patch also contained a control (biotyinylated albumin) (two dots in one row, labeled 3 in the figure). One slide (FIG. 12B) was hybridized with an anti-annexin antibody and the second with an anti-vimentin antibody (FIG. 12A). As shown in the figure, different fractions reacted with each antibody. Fractions marked 1 reacted with vimentin antibody. Four of the vimentin containing fractions were also arrayed at a ⅕ diluation, showing a commensurately diminished signal (row 4). Annexin reacted with the arrayed fractions, marked 2, obtained in the multi-dimensional liquid separation system. The experiment shows distinct patterns of reactivity based on which fractions contain which proteins.

Definitions

To facilitate an understanding of the present invention, a number of terms and phrases are defined below:

Where amino acid sequence is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding or activity with its various ligands and/or substrates.

As used herein, the term membrane receptor protein refers to membrane spanning proteins that bind a ligand (e.g., a hormone or neurotransmitter). As is known in the art, protein phosphorylation is a common regulatory mechanism used by cells to selectively modify proteins carrying regulatory signals from outside the cell to the nucleus. The proteins that execute these biochemical modifications are a group of enzymes known as protein kinases. They may further be defined by the substrate residue that they target for phosphorylation. One group of protein kinases is the tyrosine kinases (TKs) that selectively phosphorylate a target protein on its tyrosine residues. Some tyrosine kinases are membrane-bound receptors (RTKs), and, upon activation by a ligand, can autophosphorylate as well as modify substrates. The initiation of sequential phosphorylation by ligand stimulation is a paradigm that underlies the action of such effectors as, for example, epidermal growth factor (EGF), insulin, platelet-derived growth factor (PDGF), and fibroblast growth factor (FGF). The receptors for these ligands are tyrosine kinases and provide the interface between the binding of a ligand (hormone, growth factor) to a target cell and the transmission of a signal into the cell by the activation of one or more biochemical pathways. Ligand binding to a receptor tyrosine kinase activates its intrinsic enzymatic activity. Tyrosine kinases can also be cytoplasmic, non-receptor-type enzymes and act as a downstream component of a signal transduction pathway.

As used herein, the term “multiphase protein separation” refers to protein separation comprising at least two separation steps. In some embodiments, multiphase protein separation refers to two or more separation steps that separate proteins based on different physical properties of the protein (e.g., a first step that separates based on protein charge and a second step that separates based on protein hydrophobicity).

As used herein, the term “protein profile maps” refers to representations of the protein content of a sample. For example, “protein profile map” includes 1-dimensional displays of total protein expressed in a given cell. In some embodiments, protein profile maps may also display subsets of total protein in a cell. Protein profile maps may be used for comparing “protein expression patterns” (e.g., the amount and identity of proteins expressed in a sample) between two or more samples. Such comparing finds use, for example, in identifying proteins that are present in one sample (e.g., a cancer cell) and not in another (e.g., normal tissue), or are over- or under-expressed in one sample compared to the other.

As used herein, the term “separating apparatus capable of separating proteins based on a physical property” refers to compositions or systems capable of separating proteins (e.g., at least one protein) from one another based on differences in a physical property between proteins present in a sample containing two or more protein species. For example, a variety of protein separation columns and composition are contemplated including, but not limited to ion exclusion, ion exchange, normal/reversed phase partition, size exclusion, ligand exchange, liquid/gel phase isoelectric focusing, and adsorption chromatography. These and other apparatuses are capable of separating proteins from one another based on a “physical property.” Examples of physical properties include, but are not limited to, size, charge, hydrophobicity, and ligand binding affinity. Such separation techniques yield fractions or subgroups of proteins “defined by a physical property,” i.e., separated from other proteins in the sample on the basis of a difference in a physical property, but with all of the proteins in the fraction or subgroup sharing that physical property. For example, all of the proteins in a fraction may elute from a column at a defined solution condition (e.g., salt concentration) or narrow range of solution conditions, while other proteins not in the fraction remain bound to the column or elute at different solution conditions.

A “liquid phase” separating apparatus is a separating apparatus that utilizes protein samples contained in liquid solution, wherein proteins remain solubilized in liquid phase during separation and wherein the product (e.g., fractions) collected from the apparatus are in the liquid phase. This is in contrast to gel electrophoresis apparatuses, wherein the proteins enter into a gel phase during separation. Liquid phase proteins are much more amenable to recovery/extraction of proteins as compared to gel phase. In some embodiments, liquid phase proteins samples may be used in multi-step (e.g., multiple separation and characterization steps) processes without the need to alter the sample prior to treatment in each subsequent step (e.g., without the need for recovery/extraction and resolubilization of proteins).

As used herein, the term “displaying proteins” refers to a variety of techniques used to interpret the presence of proteins within a protein sample. Displaying includes, but is not limited to, visualizing proteins on a computer display representation, diagram, autoradiographic film, list, table, chart, etc. “Displaying proteins under conditions that first and second physical properties are revealed” refers to displaying proteins (e.g., proteins, or a subset of proteins obtained from a separating apparatus) such that at least two different physical properties of each displayed protein are revealed or detectable. For example, such displays include, but are not limited to, tables including columns describing (e.g., quantitating) the first and second physical property of each protein and two-dimensional displays where each protein is represented by an X, Y locations where the X and Y coordinates are defined by the first and second physical properties, respectively, or vice versa. Such displays also include multi-dimensional displays (e.g., three dimensional displays) that include additional physical properties.

As used herein, the term ion channel protein refers to proteins that control the ingress or egress of ions across cell membranes. Examples of ion channel proteins include, but are not limited to, the Na⁺-K⁺ ATPase pump, the Ca²⁺ pump, and the K+leak channel.

As used herein, the term “detection system capable of detecting proteins” refers to any detection apparatus, assay, or system that detects proteins derived from a protein separating apparatus (e.g., proteins in one or fractions collected from a separating apparatus). Such detection systems may detect properties of the protein itself (e.g., UV spectroscopy) or may detect labels (e.g., fluorescent labels) or other detectable signals associated with the protein. The detection system converts the detected criteria (e.g., absorbance, fluorescence, luminescence etc.) of the protein into a signal that can be processed or stored electronically or through similar means (e.g., detected through the use of a photomultiplier tube or similar system).

As used herein, the terms “centralized control system” or “centralized control network” refer to information and equipment management systems (e.g., a computer processor and computer memory) operably linked to multiple devices or apparatus (e.g., automated sample handling devices and separating apparatus). In preferred embodiments, the centralized control network is configured to control the operations of the apparatus and device linked to the network. For example, in some embodiments, the centralized control network controls the operation of multiple chromatography apparatus, the transfer of sample between the apparatus, and the analysis and presentation of data.

As used herein, the terms “solid support” or “support” refer to any material that provides a solid or semi-solid structure with which another material can be attached. Such materials include smooth supports (e.g., metal, glass, plastic, silicon, and ceramic surfaces) as well as textured and porous materials. Such materials also include, but are not limited to, gels, rubbers, polymers, and other non-rigid materials. Solid supports need not be flat. Supports include any type of shape including spherical shapes (e.g., beads). Materials attached to solid support may be attached to any portion of the solid support (e.g., may be attached to an interior portion of a porous solid support material). Preferred embodiments of the present invention have biological molecules such as proteins attached to solid supports. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linking group.”Such spacer molecules are molecules that have a first portion that attaches to the biological material and a second portion that attaches to the solid support. Thus, when attached to the solid support, the spacer molecule separates the solid support and the biological materials, but is attached to both.

As used herein, the term “directly bonded,” in reference to two molecules refers to covalent bonding between the two molecules without any intervening linking group or spacer groups that are not part of parent molecules.

As used herein, the terms “linking group” and “linker group” refer to an atom or molecule that links or bonds two entities (e.g., solid supports, proteins, or other molecules), but that is not a part of either of the individual linked entities.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “sample” is used in its broadest sense. In one sense, it is meant to include a specimen or culture obtained from any source, as well as biological and environmental samples. Biological samples may be obtained from plants and animals (including humans) and encompass fluids, solids, tissues, and gases. Biological samples include blood products, such as plasma, serum and the like. Environmental samples include environmental material such as surface matter, soil, water, and industrial samples. These examples are not to be construed as limiting the sample types applicable to the present invention.

### GENERAL DESCRIPTION OF THE INVENTION

The present invention relates to systems and methods for the analysis of proteins. For example, the present invention provides methods for identifying and characterizing surface membrane proteins. The present invention also provides methods and systems for arraying and analyzing proteins.

Protein tagging technologies have been available for a long time and have been utilized in a variety of applications, yet few studies have attempted to incorporate protein tagging as part of strategies to enhance sensitivity in combination with 2-D gel analysis. For example, protein radioiodination has been utilized for years, in different types of protein studies, yet few papers have been published that were based on the analysis of radioiodinated proteins in complex mixtures, when compared with the vast literature that exists for protein analysis in silver stained gels. Approaches to improve the detection of proteins by post-harvest alkylation and subsequent radioactive labeling with either (³H) iodoacetamide or ¹²⁵I have been described (Vuong et al., Electrophoresis 21:2594 [2000]). A procedure for isotopic biotinylation of cysteines in cell lysates has been developed to capture cysteine containing peptides, for their identification and quantitation by mass spectrometry (Gygi, S., et al., Nature Biotechnology 17:994 [1999]). However this approach does not target biotinylation of intact cells or selective biotinylation of surface membranes nor quantitative analysis of biotinylated proteins as opposed to individual peptides. Thus what is lacking is integrated methodology for the tagging of intact cells, tissues or organelles, followed by solubilization, purification, quantitative analysis and identification of complex mixtures of hundreds of tagged proteins in tissue or cell compartments such as surface membranes. The present invention provides such methods.

For example, the present invention provides methods of tagging membrane proteins to allow separation and/or characterization of the membrane proteins. In some such embodiments, the systems and methods of the present invention allow the characterization of rare proteins that would be undetectable if analyzed along with the entire proteome of a cell. The present invention also provides methods for arraying proteins to facilitate proteomic analysis.

In some embodiments of the present invention, membrane proteins (e.g., plasma membrane proteins) are associated with a first member of a binding pair. In such embodiments, when exposed to the second member of the binding pair (e.g., a second member attached to a solid support), the membrane proteins are bound to the second member through a binding interaction between the binding pair. Where the membrane proteins are tagged with the first member, but other cellular proteins are not, the membrane proteins may be separated and/or characterized away from non-membrane proteins.

In some embodiments, the binding pair comprises avidin and biotin. The high affinity and specificity of avidin-biotin interactions have been exploited for diverse applications in immunology, histochemistry, in situ hybridization, affinity chromatography and many other areas. Biotinylation reagents provide the “tag” that transforms poorly detectable molecules into probes that can be recognized by a labeled detection reagent. Once tagged with biotin, a molecule of interest such as an antibody, lectin, drug, polynucleotide, polysaccharide or receptor ligand can be used to probe cells, tissues, or protein blots or arrays, or complex solutions. The tagged molecule can then be detected with the appropriate avidin or anti-hapten antibody conjugate, which has been labeled with a fluorophore, fluorescent microsphere, enzyme, chromophore, colloidal gold, or other detectable moiety. Biotinylated probes are frequently combined with other probes for simultaneous, multicolor assays. Although the binding of biotin to native avidin or streptavidin is essentially irreversible, modified avidins can bind biotinylated probes reversibly, making them valuable reagents for isolation and purification of biotinylated molecules from complex mixtures. In some embodiments, the present invention employs strategies for large-scale analysis and identification of cellular proteins based on protein biotinylation.

Such aspects of the present invention stem from the notion that, because of the limited sensitivity/resolution of proteomics approaches that utilize whole cell or tissue proteins, the separate tagging and analysis of cell or tissue subfractions increases the yield of protein subsets. The surface membrane represents such a subset. For example, detailed analysis of surface membrane proteins in cancer uncovers proteins that have utility in diagnosis or that may be targeted for therapy. If from the same amount/starting material of a protein sample, multiple subsets can be quantitatively analyzed in parallel with increased sensitivity as can be achieved with biotinylation for surface membrane proteins, a substantial increment in resolution results without the need for increased sample procurement. This is an important issue, as in biomedical applications, samples are available in limited amounts. Thus, in some embodiments of the present invention for quantitative analysis of tagged surface membrane proteins, a protein sample, be it a cell population or a tissue biopsy, is divided into two fractions: a tagged (e.g., biotinylated) surface membrane protein fraction and the rest (i.e., the remaining cellular protein). The non-tagged fraction may be analyzed in its entirety or is further fractionated into multiple subsets based on specific characteristics of the proteins, (e.g., separate capture of phosphoproteins, glycosylated proteins etc.). While the tagging methodology is demonstrated for the analysis of surface membrane proteins in many of the examples described herein, additional tagging of other subcellular fractions such as mitochondria, nuclei etc. would result in substantial improvements in the quantitative analysis of such protein subsets.

The present invention further provides methods for arraying proteins. There is much interest in developing protein arrays (e.g., chips) that can assay protein abundance in a sample or that can identify protein targets of a given probe (MacBeath and Schreiber, Science, 289:1760 [2000]). In the protein chip approaches to date, a variety of ‘bait’ proteins such as antibodies have been immobilized in an array format onto specially treated surfaces. The surface is then probed with the sample of interest and only the proteins that bind to the relevant antibodies remain bound to the chip (Lueking, et al., Analytical Biochemistry, 270:103 [1999]). Such an approach represents a large-scale adaptation of enzyme-linked immunosorbent assays currently in use. Such protein chips may be probed with fluorescently labeled proteins from two different sources. The protein mixtures are labeled by different fluorophores that are mixed and their ratio provides a measure of the difference in abundance of the protein bound to the antibody between the two sources. This system is dependent on the availability of antibodies and their specificities. Antibodies that do not distinguish between different modified forms of a protein as may result from post-translational modification, have little utility for the quantitative analysis of the modified forms of a protein.

There is also substantial interest in immobilizing peptides, protein fragments or proteins onto arrays and samples such as a phage library or a patient serum, applied onto the array to determine binding to specific proteins or peptides of interest. The nature of the bound material may be determined by mass spectrometry or other means (Davies et al., Biotechniques, 27:1258 [1999]). The major problem with immobilizing proteins that represent a substantial fraction of the protein complement of a cell or a tissue is the need to develop an adequate source of such proteins. One approach is to produce recombinant proteins in bacteria, which are then purified and arrayed. In principle, procedures to produce recombinant proteins can be scaled up to allow large numbers of proteins to be produced for arraying. A problem with this approach is that recombinant proteins may lack such post-translational modifications that occur in cells that express these proteins and therefore, structurally the arrayed recombinant proteins may differ substantially from their counterparts produced in cells or found in biological fluids. It may be therefore desirable to obtain cell and tissue derived proteins for microarray analysis. However procedures have not been described for the isolation of large numbers of proteins from complex mixtures that could be using for microarraying. The ability to obtain proteins and protein fractions from different cell or tissue populations or different subcellular or tissue compartments would allow specialized microarrays containing proteins from a particular tissue or cell fraction (e.g., membrane fractions isolated by the systems and methods of the present invention) or comprehensive microarrays containing proteins obtained from different tissue or cell fractions to be made.

The present invention provides methods to separate and array proteins from cells or tissues or fractions thereof. For example, in some embodiments, a whole cell or tissue protein extract is separated into protein components based on one or more physical properties (e.g., cellular location, protein pI, size, etc.). For example, as described below, a whole cell protein extract from A549 lung adenocarcinoma cell line was separated in liquid phase into 20 fractions which were each further resolved by reverse phase chromatography into individual protein subfractions, yielding several hundred distinct protein peaks from which proteins are isolated and arrayed. Alternatively, specific protein compartments may be targeted for protein isolation and arraying. One such compartment consists of surface membrane proteins that can be tagged by the systems and methods of the present invention.

### DETAILED DESCRIPTION OF THE INVENTION

The detailed description is provided in the following sections: 1) Membrane Protein Tagging; II) Protein Analysis; and III) Protein Arraying.

I) Membrane Protein Tagging

As discussed above, in some embodiments of the present invention, membrane proteins (e.g., plasma membrane proteins) are tagged with a molecule that allows separation of the membrane proteins from other cellular proteins. The present invention is not limited by the nature of the tagging molecule. In preferred embodiments, the tagging molecule is a member of a specific binding pair. Specific binding pair refer to natural or synthetic molecules, wherein one of the pair of molecules has an area on its surface, or a cavity which specifically binds to, and is therefore defined as complementary with a particular spatial and polar organization of the other molecule, so that the pair have the property of binding specifically to each other. Examples of types of specific binding pairs are antigen-antibody, biotin-avidin, hormone-hormone receptor, receptor-ligand, enzyme-substrate, IgG-protein A, and the like. While not limited to any particular binding pair, for the purpose of illustration, the methods of the present invention are described below using the biotin-avidin binding pair.

A. Biotinylation

The biotin (strept)avidin system has been used for many years because of the extraordinary affinity that characterizes the complex formed between the vitamin biotin and the egg-white protein avidin or its bacterial relative streptavidin. An important feature of this system is that chemical modification of most targets with biotin, a small molecule, does little to change their biological or physicochemical properties such as enzyme catalysis. The success of this system is manifested by the availability of hundreds of avidin-biotin products from dozens of companies for a wide array of applications. In addition to thousands of original articles describing specific applications, there are numerous volumes, special journal issues and technical manuals devoted to the biotin-avidin system. The use of surface membrane biotinylation has been relied upon extensively for the characterization of specific antigens (Le Naour, et al, Science, 287:319 [2000]; Serru, et al., Biochem J., 340:103 [1999]; Lagaudriere-Gesbert, et al, Cell Immunol., 182:105 [1997]; Rubinstein, et al., Eur J Immunol., 27:1919 [1997]; Le Naour, et al., Leukemia, 11:1290 [1997]; Rubinstein, et al., Eur J Immunol., 26:2657 [1996]). The basic technology has been available for a quarter of a century. However integrated strategies have not been developed previously that allow the characterization of the full complement of biotinylated proteins and their identification. There are several obstacles that need to be overcome to achieve this objective. Biotinylation may not be readily targeted exclusively to the specific cell fraction of interest (e.g., cell surface membrane proteins). Other proteins may get biotinylated, which would interfere with data analysis and interpretation. It has been viewed that membrane proteins present substantial difficulty in adequately solubilizing them as a prelude to their separation and identification. Thus once membrane proteins have been tagged, solubilization issues need to be resolved. Even if adequately solubilized, such proteins may present difficulties in their separation and quantitative analysis, potentially because of their hydrophobicity, large molecular weight or unusual structural characteristics. The methodology developed during the development of the present invention allows the tagging of surface membrane proteins followed by their separation in, for example, multi-dimensional systems either as part of a mixture with other cellular proteins or after their partial enrichment or after their complete purification by affinity based techniques using, for example, avidin.

A basic component in a biotin-avidin based application is the moiety to be targeted. In the case of proteins, biotinylation is done usually via the ε-amino groups of lysines by using an N-hydroxysuccinimide (NHS) ester of a biotin analog. “NHS-biotin” reagents are available from several companies. Other types of biotinylation include reactivity with sulfhydryl or carboxyl groups or with carbohydrates. A major aspect of surface membrane protein biotinylation of the present invention is the use of non-membrane permeable biotin reagents, to prevent entry into the cell. NHS biotins are water-soluble. Biotinylation methods of the present invention may generally follow the protocols provided by commercial suppliers of NHS biotin reagents (e.g., Pierce Chemical Company, Rockford, Ill.). A distinguishing feature among NHS biotins is the extent of the spacer length. A suitable spacer for use with the present invention has a spacer length of 22 Å, although both shorter and longer spacers may be used.

Biotinylation provides an effective tool for the detection and purification of proteins. However, in order to retain the protein(s) biological activity and ligand binding properties, it is often necessary to perform a biotinylation reaction that will minimally biotinylate the proteins of interest. Such mild biotinylation reactions yield a mixture of biotinylated and unbiotinylated protein. During the development of the present invention, the extent to which biotinylation can be increased to enhance recovery without significantly altering the migration/separation properties of biotinylated proteins or their identification was investigated. Additional variables that should be considered include the concentration of biotin in solution, the incubation temperature that may need to be varied from room temperature to 4° C., as well as incubation time. The description below provides suitable conditions.

The present invention also provides an approach for the biotinylation of surface membrane proteins in tissues, as opposed to cells. Water-soluble biotin is able to diffuse through thin slices of tissue, bind to surface membranes, but not penetrate them. The approach is demonstrated as follows. Tumor cells are injected into mice to form xenotransplants. Then fresh tumor tissue is obtained from such xenotransplants, sliced into 1 mm thin sections and utilized for biotinylation. To remove as much as possible serum and blood constituents, tissue samples are washed with the biotin-labeling buffer. NHS-LC biotin (Pierce) is utilized for labeling. Biotinylated protein patterns are produced. The surface membrane protein patterns from the xenotransplanted (tumor) cells are comparable to similar patterns obtained for the same cell types cultured in vitro.

In some embodiments, more than one type of biotin label is used on one or more samples. For example, a first biotin tag may be labeled with a first label and a second biotin tag may be labeled with a second label. In some embodiments, proteins from a first cell or sample are labeled with the first tag and proteins from a second cell or sample are labeled with the second tag. This configuration allows quantitative analysis of the ratio of labels, indicating the relative amount of a protein of interest in each cell or sample. Methods for isotope labeling of affinity tags are known (e.g., Gygi et al., Nat. Biotechnol., 17:994 [1999]).

B. Solubilization

There is also a substantial literature pertaining to the solubilization of membrane proteins and the difficulties involved. Recently, there have been a number of publications reporting reagents, which improve protein solubilization prior to isoelectric focusing. While the improved solubilization possible with these reagents has increased the total number of membrane proteins able to be visualised on 2-D gels and also allowed the separation of some integral membrane proteins, some proteins have remained quite challenging to analyze (Herbert, Electrophoresis 20:660 [1999]). Recent studies of model organisms using a non-tagged protein approach have revealed that the plasma membrane is rich in extrinsic proteins but came up against two major problems: (i) few hydrophobic proteins were recovered in two-dimensional electrophoresis gels, and (ii) many plasma membrane proteins had no known function or were unknown in the database despite extensive sequencing. Several methods expected to enrich a membrane sample in hydrophobic proteins were compared. The optimization of solubilization procedures revealed that the detergent to be used depended on the lipid content of the sample. The corresponding proteomes were compared with statistical models aimed at regrouping proteins according to their solubility and electrophoretic properties. Distinct groups emerged from this analysis and the identification of proteins in each group conferred specific features to them (Santoni, et al., Electrophoresis 21:3329 [2000]). In one study, fractionation of proteins by Triton X-114 combined with solubilization with CHAPS resulted in the inability to detect certain membrane proteins on 2-DE gels. The use of C8phi for protein solubilization did not improve this result. However, after treatment of membranes with alkaline buffer, the solubilization of plasma membrane proteins with detergent C8phi permitted the recovery of these proteins in 2-D gels (Santoni, et al., Electrophoresis 20:705 [1999]). New zwitterionic reagents have improved the solubilization and analysis of membrane proteins (Chevallet, et al., Electrophoresis 19:1901 [1998]). Experiments conducted during the development of the present invention have shown that the use of cocktails containing such detergents in the solubilization of whole cell lysates has resulted in improved resolution of certain proteins, which was often accompanied by the loss of others. A particular constraint in the choice of a solubilization cocktail for the analysis of biotinylated proteins is the need to maintain the integrity of the affinity reaction with avidin. Experiments conducted during the development of the present invention have found that mild solubilization conditions such as the use of NP40 did not interfere with protein capture. Because of these difficulties, in some embodiments, the present invention provides a two-step approach for the comprehensive analysis of membrane proteins. In the first step, intact biotinylated proteins are extracted from biotinylated intact cells, tissues or organelles using a solubilization cocktail. Extracted biotinylated proteins are separated directly using 2-DE gels and visualized following blotting or alternatively, they are captured by avidin affinity capture, leading to their purification and subsequent analysis. This approach may not be effective for all membrane proteins, in particular for hydrophobic transmembrane proteins. To identify and quantitatively analyze such difficult proteins, a second step may be used in which biotinylated proteins not solubilized and recovered in step one, are subjected to partial cleavage either chemically (e.g., with cyanogen bromide) or enzymatically (e.g., with trypsin or other proteolytic enzymes). Such treatment results in the cleavage of the extramembranous biotinylated component of the transmembranous protein(s). Cleaved biotinylated polypeptides obtained in step two are visualized and purified in step one. Thus with step one, some biotinylated membrane proteins are recovered intact and with step two the remainder of biotinylated proteins are recovered as partially cleaved proteins. Alternatively step one may be bypassed altogether and biotinylated membrane proteins are processed directly according to step two and their surface membrane component recovered as partially cleaved proteins.

C. Avidin-Based Capture of Biotinylated Proteins

The avidin-biotin interaction is the strongest known noncovalent biological recognition between protein and ligand. The bond formation between biotin and tetrameric avidin is very rapid and once formed is unaffected by pH, organic solvents and denaturing agents. Binding can only be released by extreme conditions such as 6-8 M guanidine hydrochloride at a low pH. For purification of biotinylated proteins, a much more suitable alternative is the use of monomeric avidin, which retains the specificity of the biotin-avidin interaction while allowing gentle methods to be used for dissociation. A suitable method is provided in Example 1, below, although any capture separation method may be used.

D. Gel-Based Separation of Biotinylated Proteins

Once a protein compartment/fraction has been tagged, the tagged proteins, such as surface membrane, together with non-tagged proteins from other compartments, such as the rest of the cell or tissue sample, can be subjected to a separation process in their entirety. Alternatively, the affinity-captured proteins can be subjected to a separation process, separately from the non-tagged proteins. In some embodiments of the present invention, standard 2-D gel procedures are utilized to separate purified tagged proteins or tagged proteins together with non-tagged proteins from the same tissue source or the same cell population. In some embodiments, the present invention provides a modified gel-based approach wherein the concentration of the acrylamide gradient in the second dimension is reduced, in the presence of SDS, to facilitate entry of high MW surface membrane proteins.

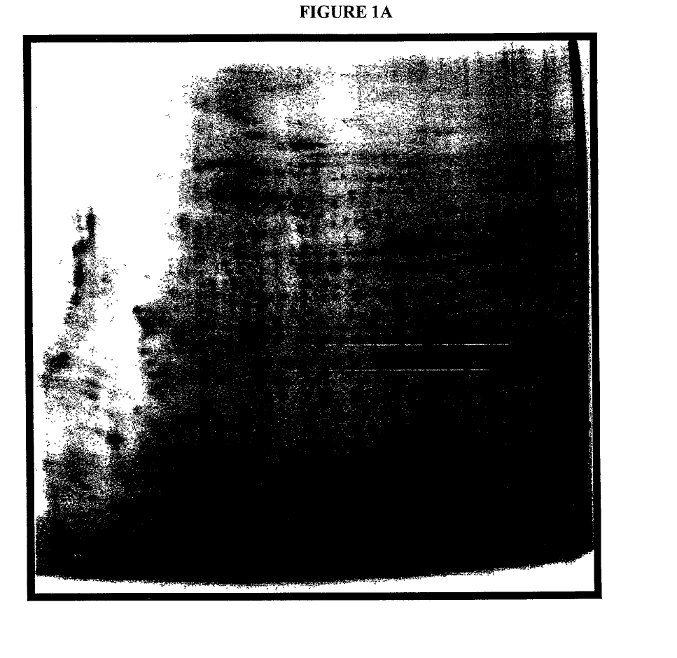

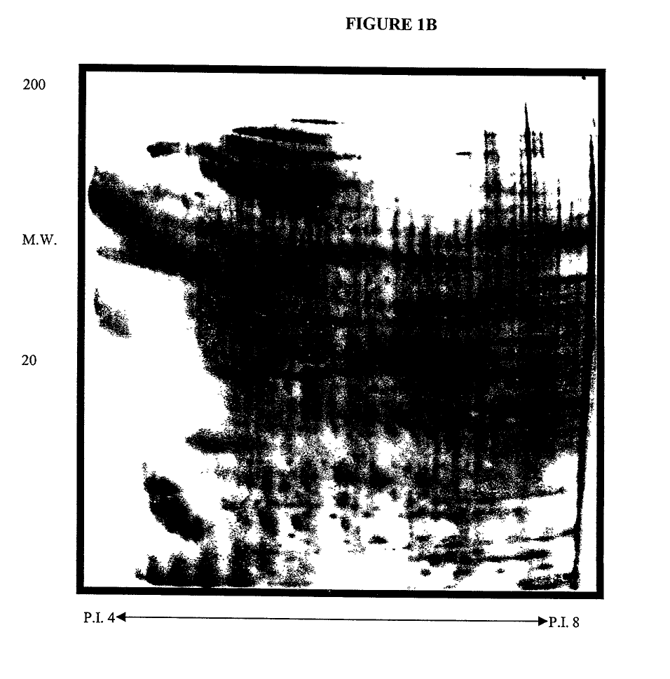

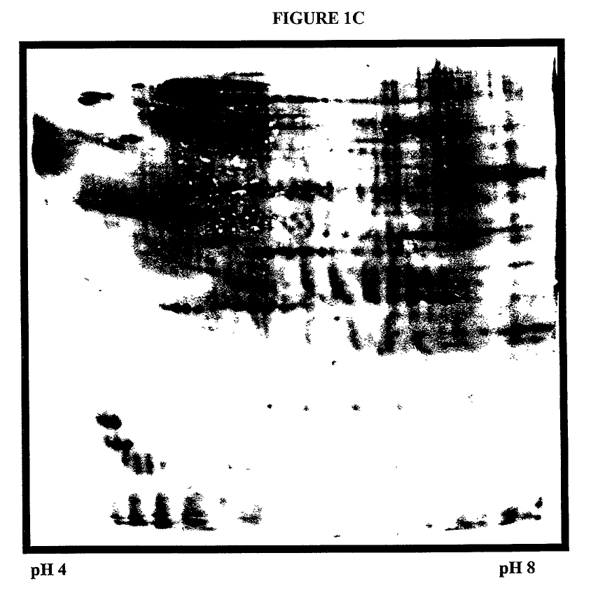

FIG. 1A shows a typical 2-D pattern of whole cells lysates from the adenocarcinoma cell line A549. First dimension separation was done using carrier ampholytes (CA), pH 4-8. Proteins were visualized by silver staining. FIG. 1B shows the 2-D pattern of the same lysate as in FIG. 1A but with visualization of only the biotinylated surface membrane proteins. The non-biotinylated proteins in the whole cell lysate are not visualized. The biotinylated proteins from lung adenocarcinoma cells were visualized after hybridization with streptavidin/horse radish peroxidase complex following transfer onto PVDF membranes. It is evident that the pattern is quite rich in separated proteins that are not visualized in silver stained 2-D gels of whole lysates, thus indicating a substantial increase in the ability to visualize and quantitatively analyze surface membrane proteins. FIG. 1C shows the same type of material as in FIG. 1B except that it was obtained from a second completely independent experiment, thus showing the remarkable reproducibility of the surface membrane protein patterns obtained by the methods of the present invention. Many of the resolved biotinylated proteins form trains of spots, as expected for membrane proteins that undergo numerous post-translational modifications (e.g., glycosylation, phosphorylation, sulphation etc.).

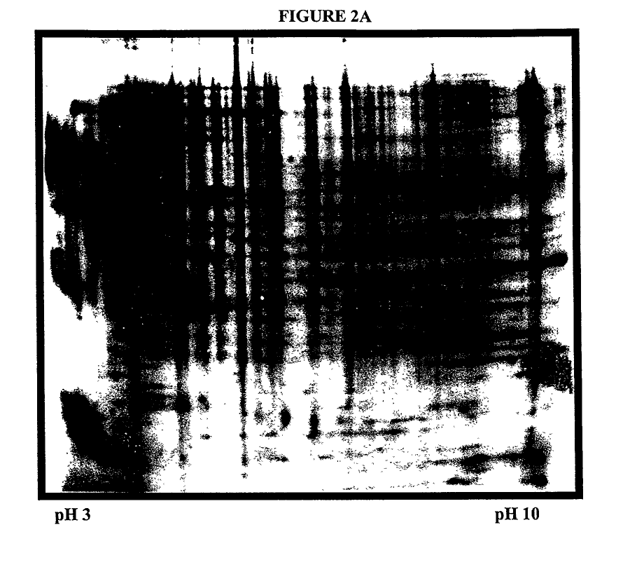

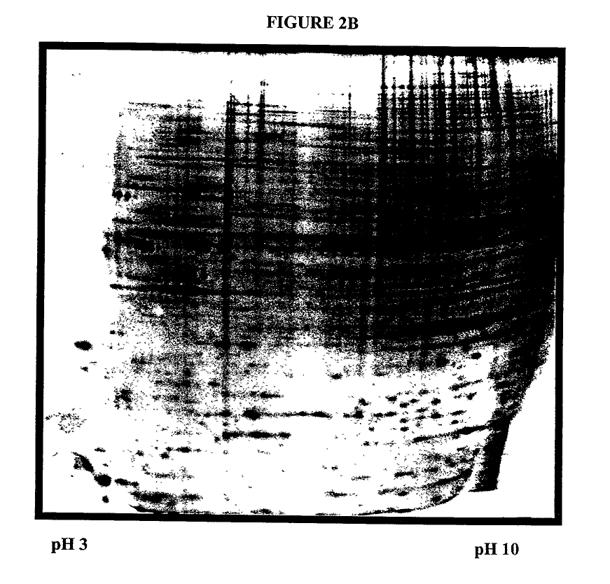

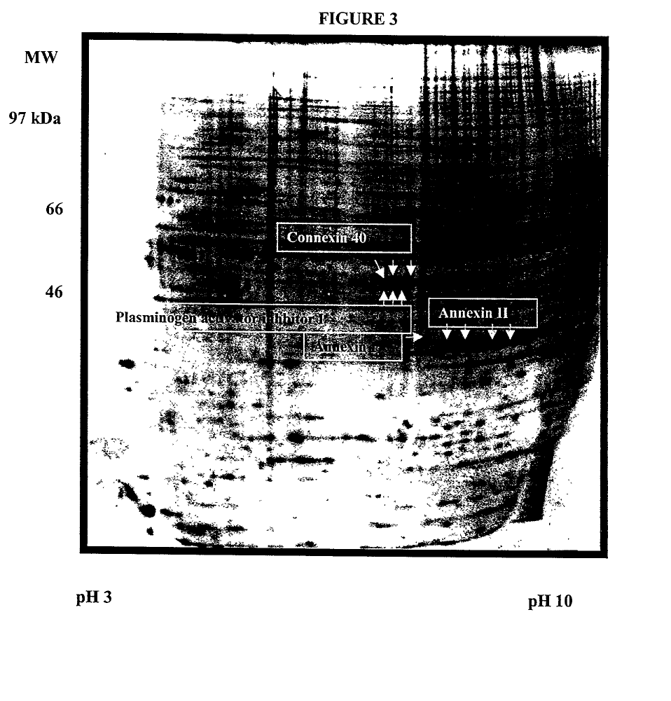

In other experiments, as shown in FIG. 2, immobilized pH gradients (IPG) pH 3-10, were utilized for first-dimension separation. FIG. 2A shows a typical IPG 2-D gel separation of A549 whole cell lysate after biotinylation of surface membrane proteins. After separation, the proteins were blotted onto PVDF membranes and the biotinylated proteins visualized as in FIG. 1. FIG. 2B shows an IPG separation of biotinylated proteins from the same source as in FIG. 2A except that whole cell proteins were passed onto an avidin column to capture the biotinylated proteins that were subsequently eluted and separately run on 2-D gels and visualized by silver staining. A remarkable similarity in the patterns in FIGS. 2A and 2B is observed. The visualization of surface membrane proteins by silver staining allows their excision from the gels for their identification by mass spectrometry or by other means for protein identification. Thus, after biotinylation of different protein sources being compared, analytical 2-D gels can be run for quantitative analysis and for identification of proteins of interest, biotinylated proteins can be purified using avidin-based affinity procedures followed by their separation using gel electrophoresis as shown here, as a prelude to their identification or using liquid based separations as shown subsequently. Examples of proteins cut from 2-D gels and subjected to identification by mass spectrometry are shown in FIG. 3. They include connexin 40, annexins I and II, and plasminogen activator inhibitor.

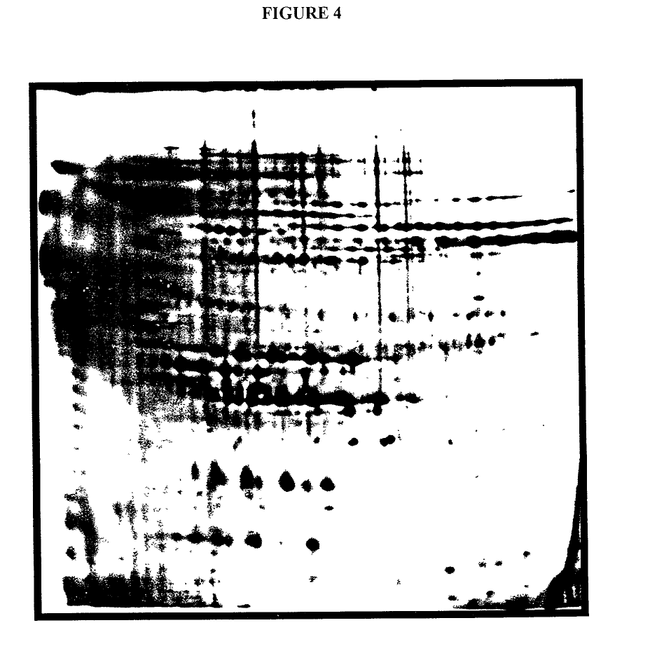

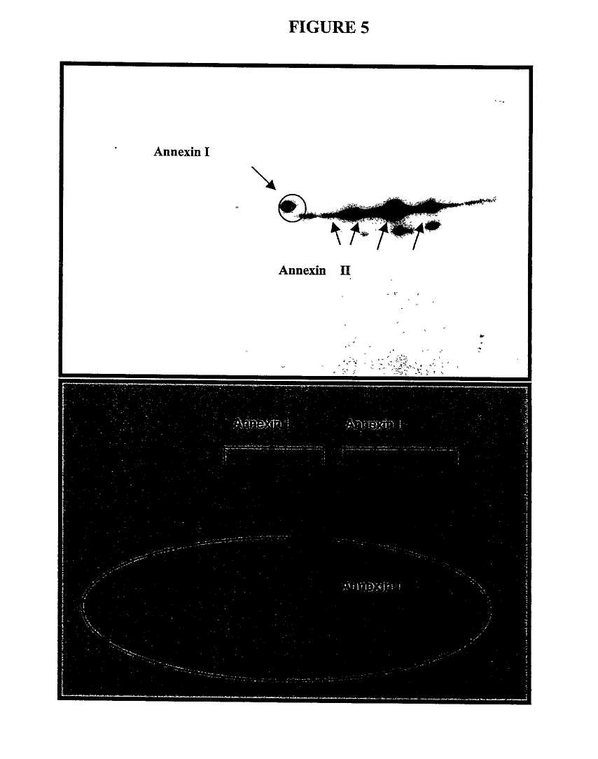

It is important to demonstrate that the patterns of surface membrane proteins quantitatively analyzed with the approach provided by the present invention did not represent just a subset of proteins with characteristics that make them easy to solubilize, easy to detect and identify but that are of only modest interest as they are ubiquitous and do not vary between cells and tissues. In other words in should be demonstrated that the patterns of surface membrane proteins that were resolved by the methods of the present invention supported the utility of this technique for biomedical applications. Therefore experiments were conducted to investigate whether cells of different lineages have detectable differences in their biotinylated surface membrane protein patterns. FIGS. 1B and 4 demonstrate differences in surface membrane protein patterns detected between two different cell lines; one is a lung adenocarcinoma and another a neuroblastoma. FIG. 5 also demonstrates how the approach uncovers biological findings of interest. In this case the demonstration that Annexin 1 forms detected on the surface membrane are different in their structure from Annexin 1 forms inside the cell. Thus, the methods of the present invention provide sensitive analysis that allows for the characterization of subtle differences between cells samples.

E. Liquid Phase Separation of Biotinylated Proteins

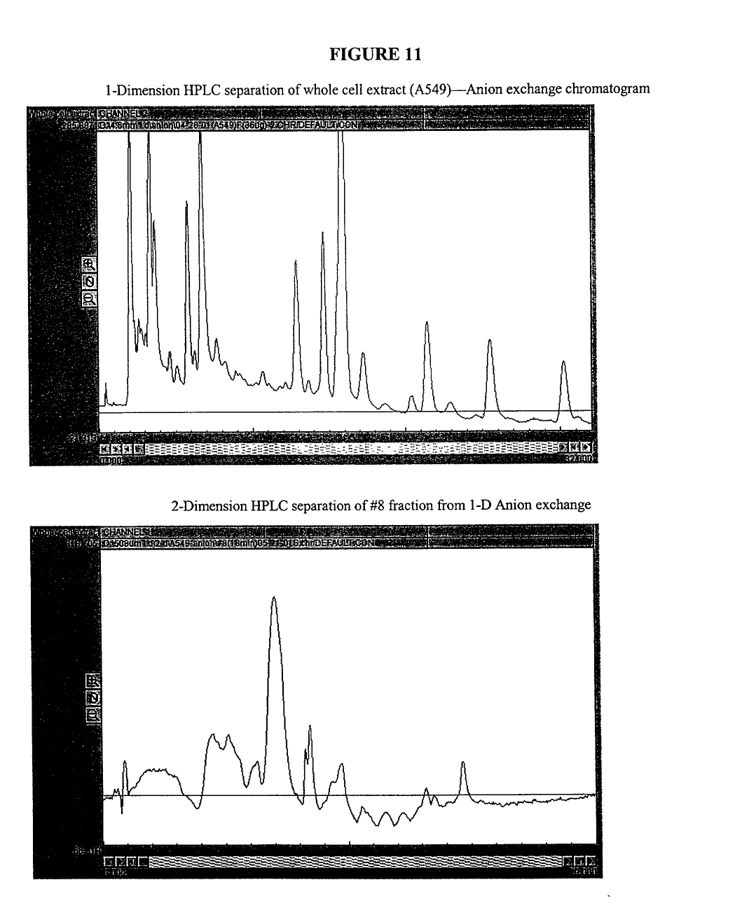

Although 2-D gels are currently the most widely used system for quantitative analysis of proteins in proteomics, they have limitations with respect to the analysis of the full complement of proteins, particularly stemming from difficulty in resolving large molecular weight and small molecular weight proteins. Liquid-based separations, including liquid based electrophoresis systems and high performance liquid chromatography have some advantages. New packing materials, columns and ultrahigh pressure pumping systems substantially improve efficiency and reduce analysis time for columns packed with small particles (MacNair et al., Anal. Chem., 69:983 [1997]). Several strategies have been implemented for comprehensive 2-D HPLC for proteome mapping (Opiteck, et al, Analytical Biochemistry 258:349 [1998]; Wagner, et al, J Chromatogr, 893: 293 [2000]; and Opiteck, et al., Anal. Chem., 69:1518 [1997]). For example, Jorgenson's group has implemented a 2-D liquid chromatographic system, which uses size-exclusion liquid chromatography followed by reversed-phase liquid chromatography to separate proteins in Escherichia coli lysates. Size-exclusion chromatography was conducted under either denaturing or nondenaturing conditions. 2D HPLC protein purification and identification system was used to isolate the src homology (SH2) domain of the nonreceptor tyrosine kinase pp6oc-src and beta-lactamase, both inserted into E. coli, as well as a number of native proteins comprising a small portion of the E. coli proteome (Opiteck, et al., Analytical Biochemistry 258:349 [1998]). The use of size-exclusion chromatography in such a system is problematic because of the limited resolution generally of such columns, requiring inordinate column length and separation time to achieve good resolution. A more suitable alternative is the use of ion exchange columns in the first dimension (Wagner, et al., J Chromatogr., 893:293 [2000]). In one configuration, cation-exchange chromatography is followed by reversed-phase chromatography. The two LC systems are coupled by a multi-port valve equipped with storage loops and under computer control. The RPLC effluent is sampled by both an UV detector and an electrospray mass spectrometer. In this way, complex mixtures of large biomolecules can be rapidly separated, desalted, and analyzed for molecular weight in less than 2 h (Opiteck, et al., Anal. Chem., 69:1518 [1997]). Other innovations include the use of capillary electrochromatography to separate proteins (See e.g., Dermaux and Sandra, Electrophoresis 20:3027 [1999]). The capture and subsequent elution in a liquid phase of biotinylated proteins are compatible with their subsequent separation in a multi-dimensional liquid based separation system. An example of a sample separated in a multi-dimensional liquid based separation system is shown in FIG. 11.

Two-dimensional liquid phase separation methods have been developed that are capable of resolving large numbers of cellular proteins. In one method, the proteins are separated by pI using isoelectric focusing in the first dimension and by hydrophobicity using reversed phase HPLC in the second dimension. Separation modes by electrophoresis include isoelectric focusing that may be accomplished using an apparatus referred to as Rotofor (Ayala et al., Applied Biochemistry and Biotechnology, 69:11 [1998]). This device allows for high protein loading and rapid separations that require four to six hours to perform. The second dimension includes reverse phase high performance liquid chromatography (HPLC). This method provides reproducible high-resolution separations of proteins according to their hydrophobicity and molecular weight. The use of non-porous silica packing material minimizes some problems associated with porosity and low recovery of larger proteins, as well as reduced analysis time.

Once biotinylated proteins have been captured using affinity based procedures, their subsequent elution and recovery in liquid medium makes them well suited for separation in a liquid based system. In some embodiments, the present invention provides a modular liquid-based system for the separation of biotinylated proteins. In this modular system, any one of several liquid separation modes in a first dimension can be combined with a liquid based separation mode in the second dimension. Alternatively, fractions obtained with a liquid separation mode can be subjected to a gel based separation mode in the second dimension. Preference is for liquid based separation modes in the final separation dimension that are compatible with current strategies for the mass spectrometric characterization of proteins.

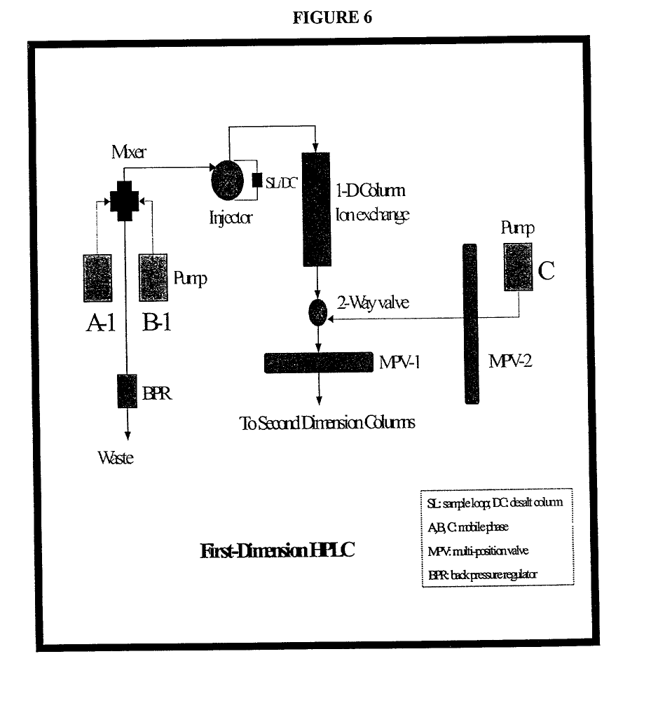

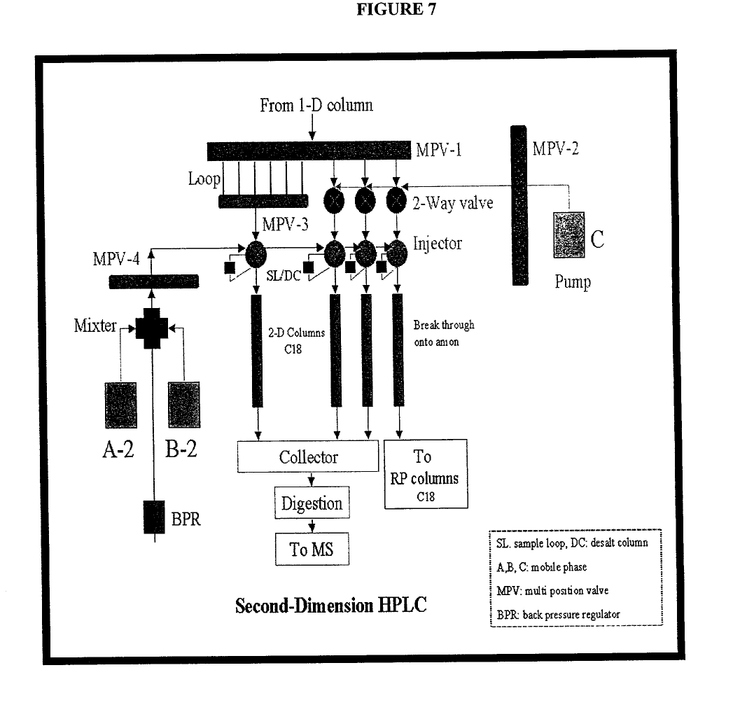

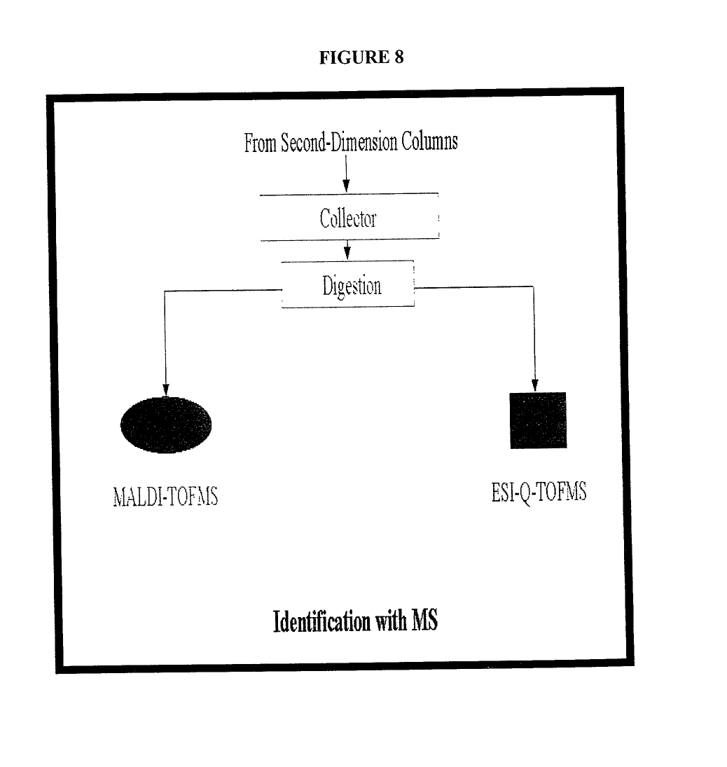

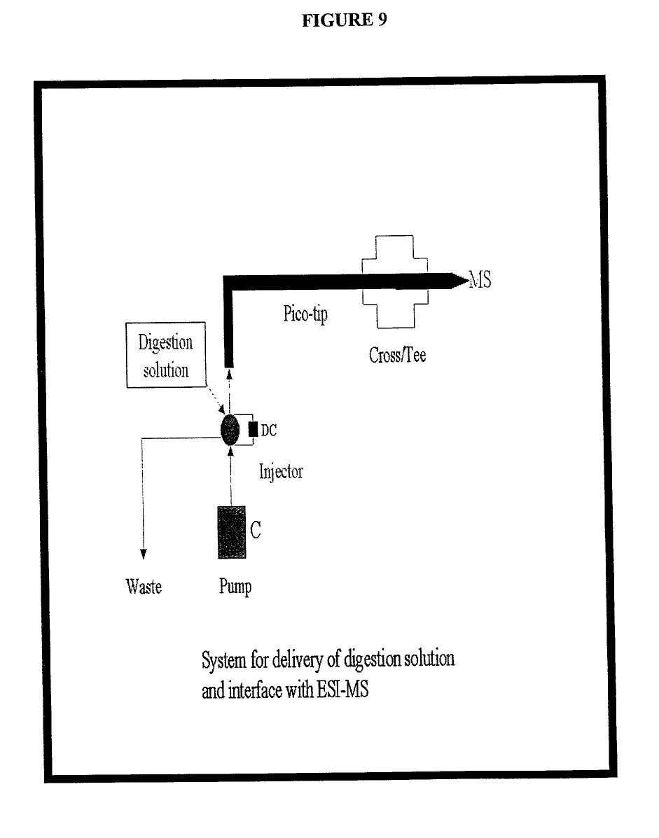

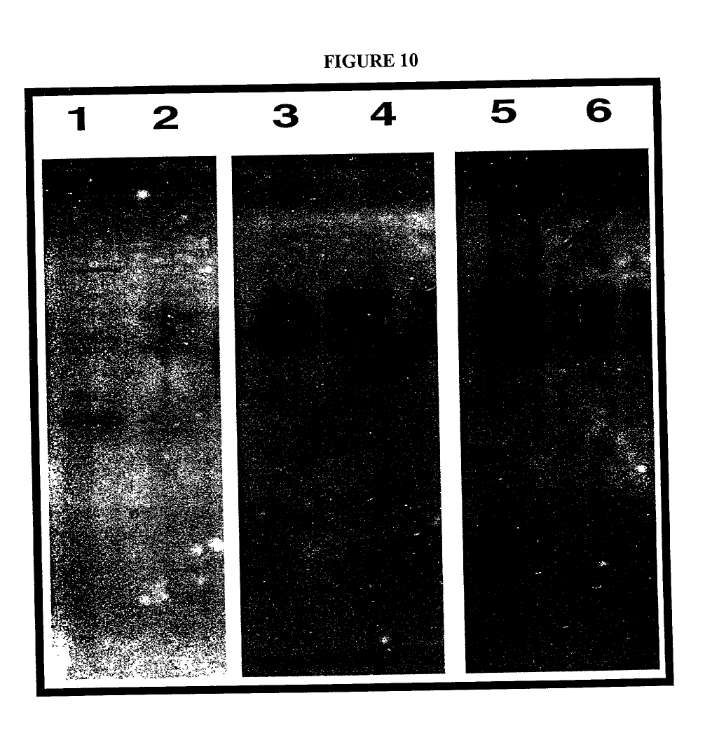

The basic principle is to implement a modular system in which different column types or media can be substituted with each other (e.g., Rotofor, or cation vs. anion vs. affinity column). The final separation is preferably accomplished using a reverse phase column. FIGS. 6-9 show chromatography-based schemes for the separation of biotinylated proteins. Fractions or peaks eluting from the first dimension are subjected to a second-dimension separation (e.g., reversed-phase chromatography) to further separate proteins. In some embodiments, breakthrough proteins not adequately fractionated in one type of separation (e.g., anion exchange) are recaptured onto an affinity column and further separated using a different mode (i.e., cation exchange instead of anion exchange) and eluted individual fractions subsequently resolved by reverse phase separation. The overall pattern obtained for separated proteins from one sample source can be compared with the pattern from another sample source. Any peak/fraction that shows interesting differences or similarities may be subjected to mass spectrometric identification or identification using other means. Alternatively all the fractions collected can be subjected to protein identification for a systematic characterization of biotinylated proteins. Thus, an aliquot of separated proteins may be deposited into 96-well microtiter plates via a fraction collector and fractions of interest are analyzed by mass spectrometry such as matrix-assisted laser desorption ionization time-of-flight mass spectrometry (MALDI-TOF/MS) and/or electrospray mass spectrometry (ESI/MS). The bulk of recovered proteins may be used for identification by tandem mass spectrometry. An example of suitable conditions for conducting such methods is provided in Example 3, below.

II) Protein Analysis

Separated proteins may be analyzed using any suitable method. Where the identity of proteins is desired, in preferred embodiments, separated proteins (e.g., membrane proteins) are subjected to mass spectrometric techniques.

In the past decade, the technology presented by new mass spectrometric methods has made the identification of proteins separated by gel electrophoresis or liquid chromatography, a much more productive endeavor. Two mass spectrometric techniques that have dramatically extended the potential of mass spectrometry for protein analysis are matrix assisted desorption ionization (MALDI) and electrospray ionization (ESI). There are on-going improvements in instrumentation that relies on these techniques that increase their throughput, sensitivity and user friendliness and data handling. Additional gains in the sensitivity of mass spectrometry methods have been achieved by improvement of sample ionization efficiency, refinement of detection techniques and the efficient use of generated of generated ions. Several illustrative uses of mass spectrometric methods of the present invention are provided below.

A) Off-Line Digestion

Mass spectrometric identification of proteins generally requires their digestion, followed by a desalting step. Using MALDI-TOF mass spectrometry, the masses of peptides derived from an in-gel proteolytic digestion are measured and searched against a computer-generated list formed from the simulated digestion of a protein database using the same enzyme. A relatively new high-resolution tandem mass spectrometry line of instrumentation, the quadrupole-time-of-flight (Q-TOF) tandem mass spectrometer, has been used for proteomic analysis that complements MALDI. Tandem mass spectrometry separates a peptide ion from a mixture of ions for dissociation. In a second step, the m/z values of the fragments are separated and detected. The combination of high-resolution two-dimensional separation of a complex mixture of proteins, followed by analysis using Q-TOF-MS/MS of trypsin-digested proteins, allows identification of a wide range of proteins. This instrument is based on ESI followed by a first quadrupole analyzer to select precursor ions, a collision-gas cell, orthogonal acceleration of the first-generation product ions plus precursor survivors, and finally high resolution time-of-flight analysis, using a reflectron System, to analyse the product ions. Complete or partial MS/MS spectra for some tryptic-digested peptides can be obtained. This allows some peptide sequences to be compared with the database, in order to assist with identification of the protein. In some preferred embodiments, instruments are used that comprise software capabilities for database searching online.

B) On-Line Analysis of Protein

Because ESI is predominantly a concentration-sensitive ionization technique, a fit exists with liquid chromatography miniaturization. Surface membrane proteins targeted for identification may be identified by LC/MS.

Flow rate: 100 nl/min˜3 μl/min, no sheath make-up

| | Sampling | | | |
|---|---|---|---|---|
| Method | type | pumped flow | Sample volume | Tip I.D. |
| Micro- | On-line | yes | 0.1 μl ˜ 10 μl | 2 μm ˜ 50 μm |
| electrospray | | | | |

Improved sensitivities are achieved when the flow rate of the solution or effluent is between 0.5 μl/min and 3 μl/min that is compatible with capillary HPLC (Markides, J. Microcolumn Separations 11:353 [1999]). The separated protein is directly analyzed with ESI-Q-TOF MS to obtain molecular weight information that allows tracking of the same protein(s) in multiple samples and experiments. With the of use micro HPLC, a splitter is used to keep the flow rate in the tip between 0.5 μl/min and 3 μl/min.

The methods described above have been used to demonstrate that 1) with biotinylation of A549 adenocarcinoma cells, the proteins visualized following blotting, represent cell surface membrane proteins; 2) with this biotinylation procedure, differences in patterns between cells of different lineages, or differentiation states, can be observed to support the utility of this approach for biomedical applications; and 3) the biotinylation approach can be adapted for the biotinylation of surface membrane proteins in tissue samples.

III) Protein Arraying

To facilitate analysis of proteins, whether they are membrane proteins tagged by the above methods or any other protein samples, proteins may be arrayed using methods of the present invention. Procedures for attaching proteins to solid surfaces are known. For example, MacBeath and Schreiber (MacBeath and Schreiber, supra) used poly-L-lysine coated slides for microarraying. Nitrocellulose coated slides are also available commercially. Exemplary attachment and arraying methods for use in the present invention are provided in Example 4. The detection of specific proteins among the arrayed samples is provided in Example 5. Arrayed proteins from A549 cell proteins lysates produced by these methods were scanned in a GeneTac LS IV scanner using a 550 nm laser. Among the large number of distinct protein fractions from A549 cell protein lysates that were arrayed, each of the proteins for which specific antibodies were utilized were detected in arraying spots from different wells. The proteins that were arrayed represented Rotofor fractions that were each further separated by reverse phase high performance liquid chromatography. Op18, vimentin, PGP 9.5, Annexin I and Annexin II were detected in distinct fractions that were spotted. Annexin I and Annexin II, that are present in the membrane protein fraction of A549, were detected in specific fractions of surface membrane proteins that were arrayed. The surface membrane proteins were obtained from A549 cells that were surface biotinylated, followed by capture of surface membrane proteins using avidin affinity columns and their subsequent separation by a combination of ion exchange and reverse phase high performance liquid chromatography.

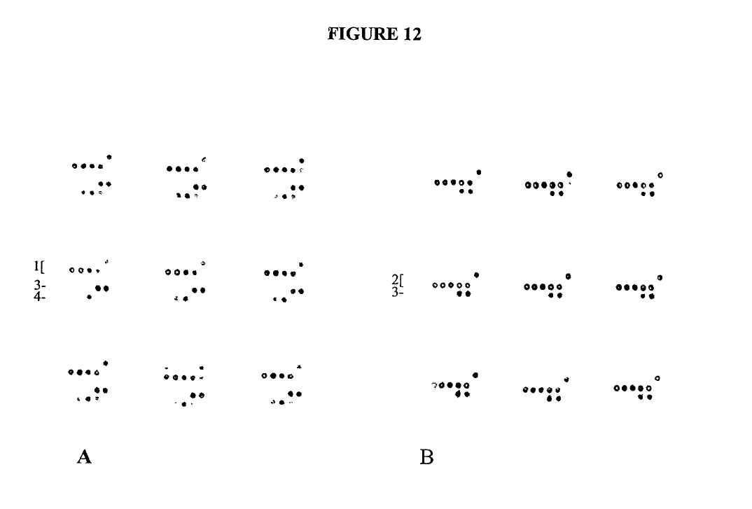

In preferred embodiments of the present invention, proteins are arrayed in physical locations on a solid support based on a physical property of the protein. For example, separated protein samples comprising subsets of total cell protein may be arrayed in specific addressed locations on an array. In some embodiments, the separated subsets of proteins comprise proteins separated by the tagging methods of the present invention. In such embodiments, the subsets of proteins comprise membrane proteins or non-membrane proteins. In other embodiments, the arrayed protein fractions are characterized by one or more physical properties. For example, proteins separated by the two-phase liquid separation methods of the present invention may be collected in fractions defined by protein size and pI. By arraying each fraction separately or independently of other fractions, proteins sharing similar physical properties are arrayed together for analysis. In some preferred embodiments, arraying is automated and linked to the protein separation procedure. For example, collected fractions from a separation apparatus may be directed to an arraying station (e.g., a 32-pin Flexys arrayer; Genomic Solutions) for spotting onto a solid support. Using these arraying methods, the present invention provides protein arrays comprising defined subsets of proteins with known addresses. This partitioning of proteins based on one or more physical properties facilitates further analysis. For example, drug candidates suspected of interacting with cell surface proteins may be targeted to arrays comprising cell membrane proteins rather then subjecting them to an array with total cell protein. An advantage of arraying only a subset of proteins is that the concentration and sensitivity of the array may be optimized for the specific protein fraction to be arrayed. For example, rare proteins may be concentrated to maximize detection, wherein their detectability amongst a total cell protein array would be questionable. An advantage of the present approach for producing protein arrays compared to an approach that relies on arraying of recombinant proteins is that the proteins being arrayed occur in the same state in which they were modified through post-translational modification as they occurred in the cells or tissues from which they were derived, whereas recombinant proteins do not reflect any such modifications. Thus the present approach for protein fractionation to yield individual proteins or protein fractions for microarraying provides the means to identify individual proteins or protein fractions that react with a variety of targets such as drugs or specific antibodies. Reactive arrayed proteins or fractions can be further investigated, identified or further resolved as they have been individually collected with one aliquot used for arrayed and another aliquot stored for any future investigations. An example of detected arrayed proteins using methods of the present invention are shown in FIG. 12.

Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); and ° C. (degrees Centigrade).

### EXAMPLE 1

### Biotinylation of Membrane Proteins

In some embodiments of the present invention, biotinylation of cell populations (e.g., colon, lung, ovarian cancer cell lines), is done using cells that are cultured under standard conditions at 37° C. in a 6% C0₂-humidified incubator in DMEM (Dubelcco's modified Eagle's medium/F12, GIBCO) supplemented with 10% fetal calf serum (GIBCO), 100 U/mL penicillin, and 100 U/mL streptomycin (Gibco-BRL, Grand Island, N.Y.). Cells are passaged weekly after they reach 70-80% confluence. A starting procedure for surface biotinylation is as follows. Cells are washed three times in Hank's buffered saline and incubated in 10 mM hepes pH 7.3, 150 mM NaCl, 0.2 mM CaCl₂, 0.2 mm MgCl₂ and 0.25 mg/ml Sulfo-NHS-LC biotin (Pierce, Rockford, Ill.) at 4° C. with gentle agitation. The reaction is quenched by washing with ice-cold PBS-Ca-Mg (pH 7.40, 0.1 mM CaCl₂ and 1 mM mgCl₂) to remove free biotin and to inhibit the reactive group. After labeling, cells are scraped in 100 μl lysis buffer (150 mM NaCl, 20 mM N-2-hydroxyethypiperazine-N′-2-ethanesulfonic acid, 1 mM EDTA, 1% Nonindet P-40 (NP40), 100 μg/ml aprotinin, 100 μg/ml leupeptin, and 2 mM phenylmethylsulfonylfluoride). The suspension is vortexed for 5 min, then sonicated in an ultrasonic water bath for 5 min and revortexed again, and incubated for 1 h at 0° C. Sonication significantly assists in solubilization of membrane proteins.

### EXAMPLE 2

### Avidin Capture of Biotinylated Proteins

Monomeric avidin from Pierce Chemical Company (Rockford, Ill.) was used for the capture of biotinylated proteins. A 3 ml column of immobilized monomeric avidin column is prepared according to the manufacturer's instructions. The column is washed with PBS, followed by a solution of 2 mM D-Biotin in PBS to block any non-reversible biotin binding sites on the column, followed by a regeneration buffer (0.1 M Glycine, pH 2.8) to remove the loosely bound biotin from the reversible biotin-binding sites and then with 2×10 ml PBS. Biotinylated lysates are applied to the column that is maintained at room temperature for 1 h to increase avidin-biotin binding. The column is then washed with PBS to remove non-biotinylated proteins from the column. The absorbance of the fractions is monitored at 280 nm until all unbound proteins have been washed off the column and the absorbance of the fractions has returned to baseline. For elution of biotinylated proteins, 0.1 M Glycine, pH 2.8 is added, and the eluent fractions are buffered with 1M Tris.HCl (pH=8.6) collected, pooled, and concentrated using a centricon Y-M 3 (Millipore, Bedford, Mass.). Again, the absorbance of the fractions is monitored at 280 nm until absorbance has returned to baseline. The column is then washed with PBS and stored with 3 ml of 0.05% NaN₃ in PBS. Elution with 0.1 M Glycine, pH 2.8 instead of 2 mM D-biotin resulted in a more concentrated fraction of eluted biotinylated proteins. Several products are available on the market with different properties and with immobilized supports of different particle size (Sigma, Promega, Pierce, Molecular Probes, PerSeptive etc.), and with different binding efficiency, selectivity and recovery.

### EXAMPLE 3

### Chromatographic Protein Separation

Systems have been assembled during the development of the present invention, including a 2-D HPLC system from individual components, which can be used for preparative LC, conventional-HPLC, Micro-HPLC, Capillary-HPLC and Nano-HPLC, in large part due to the capabilities of the pumps. The sensitivity of LC methods is a quadratic function of the LC column diameter. For a given mobile phase velocity, analyte peak volumes are proportional to peak width and the column cross-section area. Replacement of a conventional 4.6 mm internal diameter (ID) column by a 0.1 mm (ID) column yields a theoretical increase in sensitivity by a factor (4.6/0.1)²=2116, assuming equal sample volumes are utilized and provided extra-column dead volumes are minimal.

Converting the system from macro to micro-LC is accomplished by changing:

1) columns;

2) tubings;

3) the flow cell of the UV detector;

4) sample loops.

The pumps, detectors, injectors and multiple position valves need not be changed.

TABLE
requirements for macro to nano types of separations

| | | | Sampling | Detection |
|---|---|---|---|---|
| Type | Column I.D. | Flow rate | volume* | method |
| Preparative | >10 mm | −1 ml/min | −mls | Flow cell |
| LC | | | | |
| Conventional | 2.1 ˜ 7.8 mm | >0.1 ml/min | −μls | Flow cell |
| HPLC | | | | |
| Micro HPLC | 760 μm ˜ 1.0 mm | 10 ˜ 100 μl/ | <10 μl | Flow cell |
| | | min | | |
| Capillary | 150 μm ˜ | 1 ˜ 10 μl/ | <1.0 μl | On-column, |
| HPLC | 320 μm | min | | or Flow cell |
| Nano HPLC | 50 μm ˜ 100 μm | 0.1 ˜ 1.0 μl/ | <50 nl | On-column, |
| | | min | | or Flow cell |

Chromatography Conditions:

Conditions that have been utilized for anion exchange are as follows:

1. Column:

1.0 mm i.d.×150 mm L, 8μ 1000 Å (supporter materials is polystyrene divinylbenzene co-polymer, which couples with quaternised polyethyleneimine (PEI) structure having +N(CH₃)₃ as the functional group. The column is from Michrom BioResources, Inc.(Auburn, Calif.).

2. Gradient Elution:

Mobile A: 15% MeOH+10 mmol/L Tris-HAc, pH 8.05;

Mobile B: 10 mmol/L Tris-HAc+1.0 mol/L NaAc, pH 8.05.

From 0 to 5 min: 0% B;

From 5 min to 65 min: 0% B-90% B.

Sampling: 10 μl.

3. Flow Rate:

50 μl/min.

4. Detection Wavelength, 280 nm

Desalting columns are used prior to reverse phase and typically, for an analytical run, would consist of: 150 μm I.D., 2-mm length.

The starting conditions for reverse phase optimizations are as follows:

1) Dimension of the 1-D Column

150 μm I.D., 15 cm length.

2) Gradient Condition

A: 0.1% TFA (or, 0.5% acetic acid) in water

B: 0.1% TFA (or, 0.5% acetic acid) in Acetonitrile (ACN)

Flow rate: ˜5.0 μl/min, 1,500˜2,500 psi

B: 0 60% within 10 min

Detection wavelength, 280 nm

### EXAMPLE 4

### Protein Arraying

For immobilizing proteins in wells of a multi-well plate protein concentration in the wells ranged from 0.01 to 0.3 mg/ml.

Washing steps: PBS/3% non-fat milk/0.1% Tween-20 solution for 1 min, then PBS/3% non-fat milk/0.02% sodium azide o/n at 4° C.

Hybridization with antibodies or antigens labeled with Cy3/Cy5 dyes. Washing steps after hybridization included PBS/0.1% Tween-20 for 20 min and then twice in PBS and twice in ddH₂O, 5-10 min each. Spin the slides to dry.

Aldehyde treated slides may also be used for microarraying (Haab et al., Genome Biology 2:0004.1 [2001]).

Protein samples were prepared at 0.1 mg/ml in 60% PBS/40% glycerol to prevent evaporation of the nanodroplets. After a 3-hour incubation in a humid chamber at room temperature, the slides were inverted and dropped onto a solution of PBS+1% BSA for 1 min. Then, right side up in the BSA solution for 1 h, room temperature, agitation is carried out followed by hybridization with protein or small molecules also labeled with fluorescent dyes. Following incubation the slides were rinsed with PBS and then washed 3 times for 3 min each with PBS +0.1% Tween-20, then twice with PBS and centrifuged.

For arraying, separated protein fractions were loaded into 384-well plates, at 5 μl per well, at a concentration of 0.05-0.2 mg/ml and the plates spun at 1,000× g for 2 min. Using a 32-pin Flexys arrayer (Genomic Solutions) the proteins were spotted onto aldehyde-treated slides. The spacing was set up at 400 μm, and the diameter of the spots typically varied between 175-225 μm. Arrays were rinsed in a PBS/3% non-fat milk solution for 1 min to remove unbound protein. The slides were then immersed in a PBS/3% non-fat milk solution for 1 h at room temperature with agitation. The arrays were finally rinsed two times in PBS, one min each, after which they were ready for hybridization.

### EXAMPLE 5

### Array Detection

The ability to detect specific proteins among the large number of separated cell proteins was tested using antibodies against annexin I, annexin II, OP18, PGP 9.5 and vimentin. The antibodies were labeled with fluorescent Cy3-dye using monofunctional reactive dye (Amersham Pharmacia Biotech) and following the manufacturer's protocol. 20 μl of dye-labeled antibody solution was applied to the slide, which was covered with a 24×50 mm cover slip and the slide placed into a CoverWell incubation chamber (Coming) for 2 h at 4° C. into a light-protected box. The arrays were rinsed with PBS and then washed with PBS+0.1% Tween-20 solution with agitation RT, for 10 min. The slides were rinsed twice with PBS for 3 min each and then rinsed twice in H₂O for 3 min each, all the washing steps at RT. Centrifugation at 200× g for 1 min let them dry ready to scan.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for identifying cell surface proteins comprising:
a) providing:
i) a sample comprising one or more cells, said cells comprising surface proteins and intracellular proteins; and
ii) a non-membrane-permeable label;
b) exposing said sample to said label under condition such that said label binds to said surface proteins to generate labeled surface proteins; and
c) identifying two or more of said labeled proteins.

2. The method of claim 1, wherein said identifying comprises identifying ten or more of said labeled proteins.

3. The method of claim 1, wherein said two or more identified labeled proteins comprise a first protein from a first class of proteins and a second protein from a second class of proteins, said first class and said second class of proteins being different protein classes selected from the group consisting of proteins kinases, growth factors, protein phosphatases, ion channels, and receptors.

4. The method of claim 3, wherein said first protein class is growth factors and said second class is not growth factors.

5. The method of claim 1, wherein prior to said identifying step, said labeled surface proteins are separated from said intracellular proteins.

6. The method of claim 1, wherein said label comprises biotin.

7. The method of claim 6, wherein said biotin comprises NHS-biotin.

8. The method of claim 6, wherein prior to said identifying step, said labeled surface proteins are separated from said intracellular proteins.

9. The method of claim 8, wherein said separating comprises binding said labeled surface proteins to a solid support comprising avidin.

10. The method of claim 1, wherein said identifying two or more of said labeled proteins comprises mass spectrally analyzing said labeled proteins.

11. The method of claim 1, further comprising the step of quantitating an amount of at least one of said two or more identified labeled proteins.

12. The method of claim 11, further comprising comparing said amount of said at least one of said two or more identified labeled proteins to an amount of said at least one of said two or more identified labeled proteins from a different cell sample.

13. The method of claim 1, wherein prior to said identifying step, said labeled surface proteins are solubilized in a buffer.

14. The method of claim 13, wherein prior to said identifying step and following said solubilizing step, said unsolubilized labeled proteins are digested.

15. The method of claim 1, wherein said one or more cells comprise cancer cells.

16. A method for characterizing cell surface proteins comprising:
a) providing:
i) a sample comprising one or more cells, said cells comprising surface proteins and intracellular proteins; and
ii) a non-membrane-permeable label;
b) exposing said sample to said label under condition such that said label binds to said surface proteins to generate labeled surface proteins;
c) separating said labeled surface proteins from said intracellular proteins to generate separated surface proteins; and
d) attaching said separated surface proteins to a solid support.

17. A method for arraying proteins, comprising:
a) providing:
i) a solid support;
ii) a sample comprising cellular proteins;
iii) a separation apparatus that separates proteins based on a first physical property;
b) treating said sample with said separation apparatus to produce a plurality of protein fractions;
c) attaching proteins from one of said protein fractions to a pre-selected location on said solid support.

18. The method of claim 17, wherein said sample comprises a cell extract from a cancer cell.

19. The method of claim 17, wherein said separation apparatus separates proteins based on protein charge.

20. The method of claim 17, wherein said separation apparatus separates proteins based on protein size.
